(19) United States
(12) Plant Patent Application Publication
(10) Pub. No.: US 2008/0141405 P1
(43) Pub. Date: Jun. 12, 2008

(54) Strawberry plant named 'PS-5096'

(75) Inventors: Stephen M. Ackerman, Salinas, CA (US); Steven D. Nelson, Watsonville, CA (US); Michael D. Nelson, Watsonville, CA (US)

Correspondence Address: FOLEY AND LARDNER LLP;SUITE 500, 3000 K STREET NW, WASHINGTON, DC, 20007, US

(73) Assignees: PLANT SCIENCES, INC.; BERRY R&D, INC.

(21) Appl. No.: 11/730,332

(22) Filed: Mar. 30, 2007

Related U.S. Application Data
(60) Provisional application No. 60/852,999, filed on Oct. 20, 2006.

(51) Int. Cl. A01H 5/00

(52) U.S. Cl. PLT/208

(57) ABSTRACT

This invention relates to a new and distinct variety of strawberry plant named ‘PS-5096’. This new strawberry plant named ‘PS-5096’ is primarily adapted to the growing conditions of the central coast of California, and is characterized by medium to small sized fruit which are orange-red to red in color; medium to small calyx diameter; conical, glossy fruit with the calyx which tends to be slightly reflexed; light to medium green foliage, which is slightly concave to slightly convex in shape with medium interveinal blistering and gloss; and position of fruiting truss tends to be more level with to above the plant with weak pubescence.

### LATIN NAME OF THE GENUS AND SPECIES OF THE PLANT CLAIMED

Fragaria ananassa

### VARIETY DENOMINATION

‘PS-5096’

### BACKGROUND OF THE INVENTION

The present invention relates to a new and distinct strawberry variety designated as ‘PS-5096’. This new variety is a result of a controlled cross made by the inventors Stephen M. Ackerman, Steven D. Nelson, and Michael D. Nelson, in 1997 between strawberry variety ‘PS-592’ (patented, U.S. Plant Pat. No. 9,903) and strawberry variety designated ‘PS-1150’ (patented, U.S. Plant Pat. No. 10,780). The variety is botanically known as Fragaria ananassa.

The seedling resulting from the aforementioned cross was asexually propagated by stolons in a nursery located in Siskiyou County, Calif., and was subsequently selected by the inventors from a controlled breeding plot in Salinas, Calif., in 1999. After its selection, the new variety was further asexually propagated by stolons in both Siskiyou County, Calif. and San Joaquin County, Calif. The new variety was extensively tested over the next several years in fruiting fields of Salinas, Calif. This propagation has demonstrated that the combination of traits disclosed herein as characterizing the new variety are fixed and remain true to type through successive generations of asexual reproduction.

### BRIEF SUMMARY OF THE INVENTION

‘PS-5096’ is primarily adapted to the climate and growing conditions of the central coast of California. This region provides the necessary winter temperatures required for it to produce a strong vigorous plant and to remain in fruit production from April through November. The nearby Pacific Ocean provides the needed humidity and moderate temperatures to maintain fruit quality during the spring and summer production months.

The following traits have been repeatedly observed and are determined to be unique characteristics of ‘PS-5096’, which in combination distinguish this strawberry plant as a new and distinct variety:

1. Medium to small sized fruit;

2. Orange-red to red fruit color;

3. Medium to small calyx diameter;

4. Conical, glossy fruit with the calyx which tends to be slightly reflexed;

5. Light to medium green foliage; slightly concave to slightly convex in shape with medium interveinal blistering and gloss; and

6. Position of fruiting truss tends to be more level with to above the plant with weak pubescence.

The strawberry varieties that are believed to be most closely related to the new strawberry variety ‘PS-5096’ are the strawberry variety ‘PS-592’ (patented, U.S. Plant Pat. No. 9,903) and the strawberry variety ‘PS-1 150’ (patented, U.S. Plant Pat. No. 10,780).

In comparison to the similar strawberry varieties ‘PS-592’ and ‘PS-1 150’, the new strawberry variety ‘PS-5096’ differs by the following combination of characteristics as described in Table 1:

TABLE 1

| | | | ‘PS-592’ | ‘PS-1150’ |
|---|---|---|---|---|
| Characteristic | | ‘PS-5096’ | (US PP9,903) | (US PP10,780) |
| 1. | Average berry weight (gm) | 19.3 | 23.1 | 17.6 |
| 2. | Achenes per berry | 254 | 304 | 293 |
| 3. | Distribution of red color | Only marginal | Marginal & central | Marginal & central |
| | of the internal flesh | | | |
| 4. | Number of crowns per Plant | 5.5 | 5.0 | 5.0 |
| 5. | Plant Size | Medium to large | Large | Medium |
| 6. | Stolon anthocyanin intensity | Absent or very weak | Medium | Medium to strong |
| 7. | Stolon pubescence | Weak to medium | Medium | Medium to strong |
| 8. | Foliage Color (upper surface) | Light to medium green | Medium green | Medium green |
| 9. | Foliage interveinal blistering | Medium | Medium to strong | Weak to medium |
| 10. | Foliage gloss | Medium | Strong | Weak |
| 11. | Fruiting truss position | Level with to above | Level with to beneath | Above |
| | relative to foliage | | | |
| 12. | Fruiting truss pubescence | Weak | Medium | Medium |

For identification a series of molecular markers have been determined for this new variety.

### BRIEF DESCRIPTIONS OF THE DRAWINGS

The accompanying color photographs illustrate the overall appearance of typical specimens of the new strawberry variety ‘PS-5096’, at various stages of development as true as it is reasonably possible with color reproductions of this type. Color in the photographs may differ slightly from the color value cited in the botanical description which accurately describe the color of ‘PS-5096’. The depicted plant and plant parts of the new strawberry variety ‘PS-5096’ were taken in Salinas, Calif., and are approximately 8 to 9 months old.

FIG. 1 shows typical fruiting field characteristics taken in the month of June, 2006.

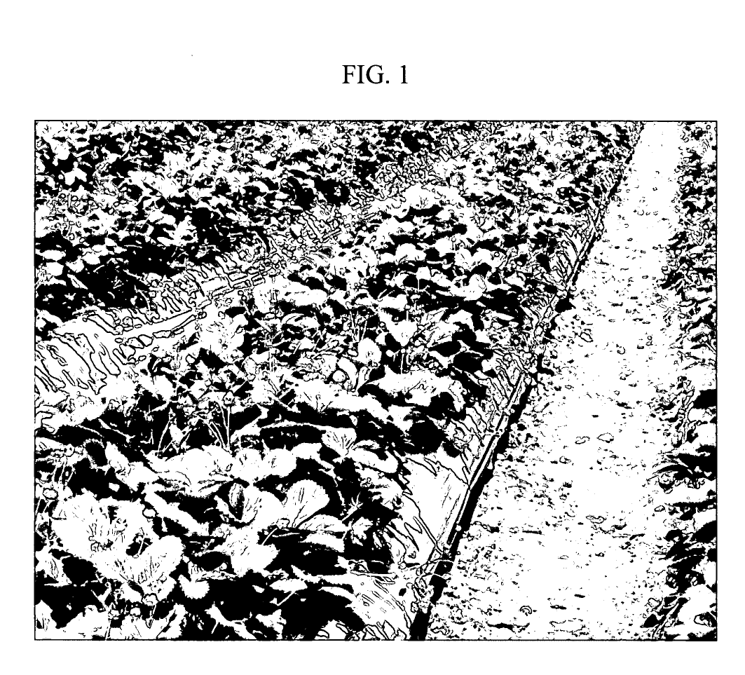

FIG. 2 shows a close-up view of typical leaf structure taken in the month of June, 2006.

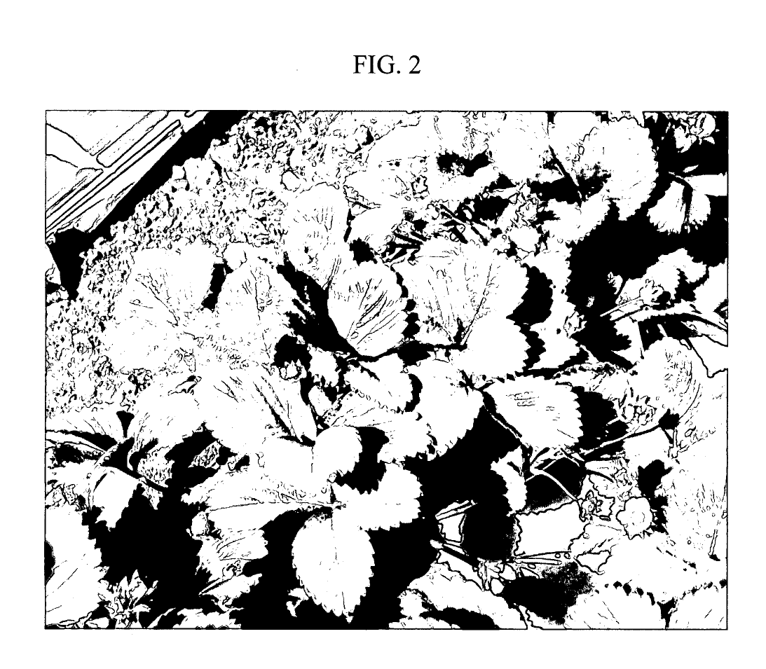

FIG. 3 shows typical mature and immature field fruit taken in the month of June, 2006.

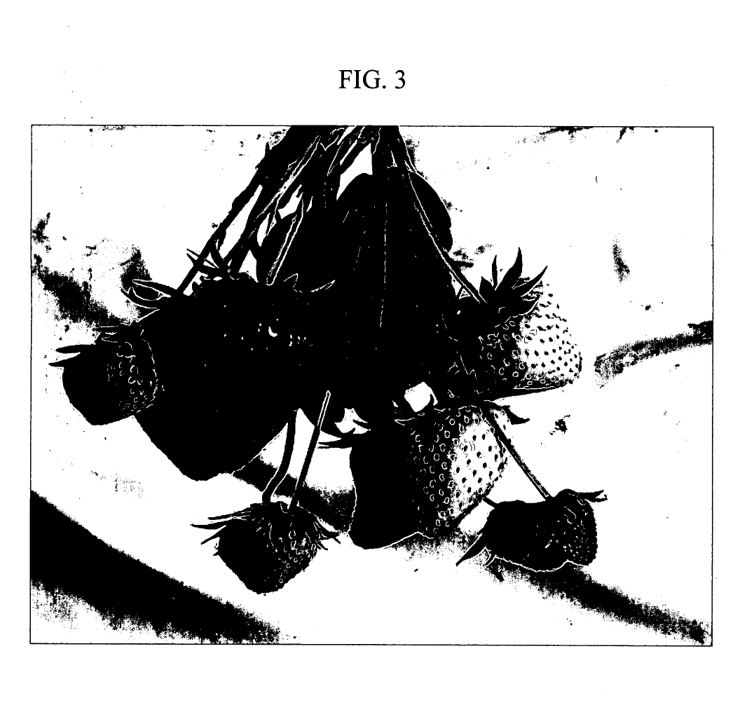

FIG. 4 shows a close-up view of fruit taken in the month of July, 2006.

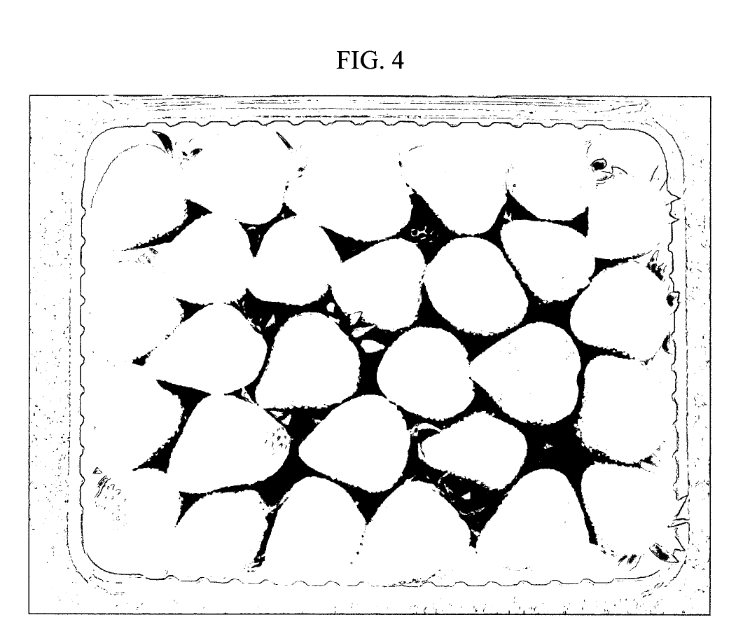

### DETAILED BOTANICAL DESCRIPTION

‘PS-5096’ has not been observed under all possible environmental conditions. The characteristics of the new variety may vary in detail, depending upon variations in environmental factors, including weather (temperature, humidity and light intensity), day length, soil type and location.

The aforementioned photographs, together with the following description of the new variety ‘PS-5096’, unless otherwise noted, is based on observations taken during the 2006 growing season in Salinas, Calif. These measurements and ratings were taken from plants of ‘PS-5096’ dug from a high-elevation nursery located in Siskiyou County, Calif. during the middle of October 2005 and planted approximately 3 weeks later in Salinas, Calif. The approximate age of the observed plants is 7 to 10 months. Yield observations and fruit quality characteristics are averaged from four years of data collected from the 2003 through 2006 growing seasons. Flower measurements and characteristics are from secondary flowers unless otherwise noted. Fruit characteristics and measurements are from secondary fruit unless otherwise noted.

Color terminology where noted follows the Munsell Book of Colors, Munsell Color, Baltimore, Md. (1976).

The following Tables 2-8 describe fruit, plant, stolon, foliage, fruiting truss, flower and pest/disease characteristics of the new strawberry ‘PS-5096’ in comparison to the similar strawberry varieties ‘PS-592’ and ‘PS-1 150’.

TABLE 2
FRUIT CHARACTERISTICS

| | | ‘PS-592’ | ‘PS-1150’ |
|---|---|---|---|
| Characteristic | ‘PS-5096’ | (US PP9,903) | (US PP10,780) |
| Color of mature fruit | 7.5R 3/12-4/12 | 7.5R 3/12-4/12 | 7.5R 4/10-3/8 |
| | Orange red to red | Orange red to red | Red |
| Color of internal flesh | 7.5R 4/10-4/12 | 7.5R 5/10-4/10 | 7.5R 4/10-4/12 |
| | Medium red | Light red | Medium red |
| Length (cm) | 43.9 | 48.3 | 42.0 |
| Width (cm) | 36.8 | 42.6 | 36.9 |
| Ratio length/width | 1.19 | 1.13 | 1.14 |
| | Slightly longer | Slightly longer | Slightly longer |
| | than broad | than broad | than broad |
| Calyx diameter (cm) | 4.0 | 5.0 | 4.2 |
| Average weight (gm) | 19.3 | 23.1 | 17.6 |
| Achene color | 5Y 6/8-7.5R 3/8 | 5Y 6/8-7.5R 3/8 | 5Y6/8-7.5R 3/8 |
| Achenes per berry | 254 | 304 | 293 |
| Achene weight (mg) | 0.59 | 0.61 | 0.56 |
| Marketable yield (gm/plt) | 1,219 | 1,392 | 1,010 |
| Size | Medium to small | Medium to large | Small |
| Predominant shape | Conical | Conical | Conical |
| Difference in shapes | None or very slight | Moderate | Slight |
| between primary and | | | |
| secondary fruit | | | |
| Band without achenes | Absent or very | Absent or very | Absent or very |
| | narrow | narrow | narrow |
| Unevenness of surface | Absent or very weak | Weak | Absent or very |
| Evenness of color | Even | Slightly uneven | Even |
| | | to even | |
| Glossiness | Strong | Medium to strong | Strong |
| Insertion of achenes | Level with surface | Level with surface | Level with |
| surface | | | |
| Insertion of calyx | In the basin to level | Level to above fruit | In the basin to level |
| Attitude of the calyx | Spreading to | Spreading to | Collapsing to |
| | reflexed | reflexed | reflexed |
| Size of calyx in relation to | Slightly larger | Slightly larger | Slightly larger |
| fruit diameter | | | |
| Adherence of calyx | Strong | Strong | Strong |
| Firmness of skin | Strong | Medium | Strong |
| Firmness of flesh | Firm | Medium | Medium to firm |
| Distribution of red color | Only marginal | Marginal and | Marginal and |
| of the flesh | | central | central |
| Hollow center expression | Moderate | Moderate to strong | Moderate to strong |
| Flavor | Good | Very good | Fair |
| Soluble solids (% Brix) | 8.3 | 8.0 | 7.4 |
| Time of first flowering | Medium | Early | Late |
| Time of first harvesting | Medium | Early | Late |
| Harvest period | Early April to | Late March to | Mid April to |
| | early December | early December | early December |
| Type of bearing | Partially remontant | Partially remontant | Partially |
| | | | remontant |

TABLE 3
PLANT CHARACTERISTICS

| | | ‘PS-592’ | ‘PS-1150’ |
|---|---|---|---|
| Characteristic | ‘PS-5096’ | (US PP9,903) | (US PP10,780) |
| Height (cm) | 29.7 | 31.0 | 30.0 |
| Spread (cm) | 31.7 | 35.1 | 28.8 |
| Crowns/plant | 5.5 | 5.0 | 5.0 |
| Size | Large to medium | Large | Medium |
| Habit | Globose | Globose | Globose |
| Density | Medium | Open to medium | Medium |
| Vigor | Medium to strong | Strong | Medium |

TABLE 4
STOLON CHARACTERISTICS

| | | ‘PS-592’ | ‘PS-1150’ |
|---|---|---|---|
| Characteristic | ‘PS-5096’ | (US PP9,903) | (US PP10,780) |
| Average number | 0.7 | 1.2 | 0.8 |
| per plant | | | |
| Fruiting Field | Few | Few to medium | Few |
| Anthocyanin | 7.5R 6/6-6/4 | 7.5R 5/6-5/4 | 7.5R 4/6-5/6 |
| coloration | | | |
| Anthocyanin | Absent or very | Medium | Medium to strong |
| intensity | weak | | |
| Diameter at bract | 4.0 | 4.4 | 4.2 |
| (mm) | Thin to medium | Thick | Thin to medium |
| Pubescence | Weak to medium | Medium | Medium to strong |

TABLE 5
FOLIAGE CHARACTERISTICS

| | | ‘PS-592’ | ‘PS-1150’ |
|---|---|---|---|
| Characteristic | ‘PS-5096’ | (US PP9,903) | (US PP10,780) |
| Foliage: | | | |
| Color of upper surface | 7.5GY 4/4-3/4 | 7.5GY 4/4-3/4 | 7.5GY 4/4-3/4 |
| | Light to medium green | Medium green | Medium green |
| Color of under side | 5GY 5/4-6/4 | 5GY 5/4-6/4 | 5GY 5/4-6/4 |
| | Light to medium | Light to medium | Light to medium |
| | grey green | grey green | grey green |
| Shape in cross section | Slightly concave | Slightly concave | Slightly concave |
| | to slightly convex | | to slightly convex |
| Interveinal blistering | Medium | Medium to strong | Weak to medium |
| Glossiness | Medium | Strong | Weak |
| Number of leaflets | 3 | 3 | 3 to 4 |
| Terminal Leaflet: | | | |
| Length (cm) | 9.2 | 9.5 | 7.2 |
| Width (cm) | 6.6 | 8.0 | 6.4 |
| Length/width ratio | 1.39 | 1.19 | 1.13 |
| Serrations/leaf | 20.7 | 20.7 | 19.3 |
| Size | Much longer | Much longer | Longer than broad |
| | than broad | than broad | |
| Shape of base | Acute | Acute | Obtuse |
| Shape of teeth | Obtuse | Obtuse | Obtuse |
| Petiole: | | | |
| Length (cm) | 20.8 | 21.1 | 19.0 |
| Diameter (mm) | 3.6 | 4.7 | 3.1 |
| Petiolule length (mm) | 13.0 | 15.7 | 9.3 |
| Pubescence | Moderate to sparse | Moderate to sparse | Moderate to |
| | | | sparse |
| Attitude of hairs | Slightly outward | Strongly outward | Strongly outward |
| Size bract leaflets | Small | Small to medium | Small |
| Stipules: | | | |
| Length (mm) | 15.4 | 20.8 | 14.5 |
| Width (mm) | 8.3 | 8.5 | 8.4 |
| Anthocyanin coloration | Medium | Medium | Absent or very |
| | | | weak |
| Color | Light to medium | Light to medium | Light to medium |
| | green | green | green |

TABLE 6
FRUITING TRUSS CHARACTERISTICS

| | | ‘PS-592’ | ‘PS-1150’ |
|---|---|---|---|
| Characteristic | ‘PS-5096’ | (US PP9,903) | (US PP10,780) |
| Length (cm) | 35.5 | 37.3 | 38.1 |
| Position relative to | Level with to | Beneath to | Above |
| foliage | above | level with | |
| Pubescence | Weak | Medium | Medium |
| Anthocyanin intensity | Light | Light | Moderate |
| Attitude at first pick | Prostrate to | Prostrate to | Prostrate to |
| | erect | erect | erect |

TABLE 7
FLOWER CHARACTERISTICS

| | | ‘PS-592’ | ‘PS-1150’ |
|---|---|---|---|
| | | (US | (US |
| Characteristic | ‘PS-5096’ | PP9,903) | PP10,780) |
| Petal color | N9.5/90.0% R | N9.5/90.0% R | N9.5/90.0% R |
| | to | to | to |
| | N9.25/84.2% R | N9.25/84.2% R | N9.25/84.2% R |
| | White | White | White |
| Corolla diameter | 28.0 | 30.6 | 30.5 |
| (mm) | | | |
| Calyx diameter | 32.0 | 38.8 | 34.0 |
| (mm) | | | |
| Petal length (mm) | 11.0 | 12.1 | 12.5 |
| Petal width (mm) | 10.1 | 11.5 | 10.8 |
| Petal length/width | 1.09 | 1.05 | 1.15 |
| ratio | | | |
| Petals/flower | 6 | 6.0 | 6.2 |
| Sepal length | 12.8 | 15.7 | 13.1 |
| (mm) | | | |
| Sepal width (mm) | 4.8 | 6.2 | 5.4 |
| Sepal length/width | 2.69 | 2.54 | 2.42 |
| ratio | | | |
| Sepals/flower | 12.1 | 11.9 | 12.4 |
| Size of calyx relative | Larger | Larger | Larger |
| to corolla | | | |
| Size of inner calyx | Same | Smaller to | Smaller to |
| relative to outer | | same | same |
| calyx | | | |
| Relative position of | Overlapping | Overlapping | Overlapping |
| petals | | | |

TABLE 8
PEST AND DISEASE REACTIONS

| | | ‘PS-592’ | ‘PS-1150’ |
|---|---|---|---|
| Characteristic | ‘PS-5096’ | (US PP9,903) | (US PP10,780) |
| Two spotted spider mite | Moderately | Moderately | Moderately |
| | susceptible | susceptible | susceptible |
| Lygus bug | Susceptible | Susceptible | Susceptible |
| Flower thrips | Moderately | Moderately | Moderately |
| | susceptible | susceptible | susceptible |
| Powdery mildew | Moderately | Moderately | Moderately |
| | susceptible | susceptible | susceptible |
| Botrytis fruit rot | Moderately | Moderately | Moderately |
| | susceptible | susceptible | susceptible |
| Angular leaf spot | Moderately | Moderately | Moderately |
| | susceptible | susceptible | susceptible |
| Virus complex | Unknown | Unknown | Unknown |

We claim:

1. A new and distinct strawberry plant named ‘PS-5096’, as herein described and illustrated by the characteristics set forth above.
